(19) United States
(12) Patent Application Publication
(10) Pub. No.: US 2023/0215579 A1
(43) Pub. Date: Jul. 6, 2023

(54) SYSTEM AND METHOD TO COMBINE MULTIPLE PREDICTIVE OUTPUTS TO PREDICT COMPREHENSIVE AKI RISK

(71) Applicant: KONINKLIJKE PHILIPS N.V., EINDHOVEN (NL)

(72) Inventors: Erina Ghosh, Cambridge, MA (US); Emma Holdrich Schwager, Cambridge, MA (US); Larry James Eshelman, Briarcliff Manor, NY (US); Kianoush Kashani, Eindhoven (NL)

(21) Appl. No.: 18/093,843

(22) Filed: Jan. 6, 2023

Related U.S. Application Data
(60) Provisional application No. 63/296,973, filed on Jan. 6, 2022.

(51) Int. Cl. G16H 50/30; G16H 10/60

(52) U.S. Cl. CPC G16H 50/30; G16H 10/60

(57) ABSTRACT

Systems, apparatuses, and methods provide for the monitoring and/or management of acute kidney injury (AKI). For example, an apparatus (100) is configured to determine whether a baseline AKI risk prediction is above a baseline threshold based on patient demographic data and patient medical history data, and perform a continuous AKI risk prediction. The continuous AKI risk prediction includes determining whether an any risk of AKI prediction is above an any AKI threshold based on dynamic intervention data and/or dynamic patient condition data, and determining an AKI stage prediction in response to a determination that the any risk of AKI prediction is above the any AKI threshold based on the dynamic intervention data and/or the dynamic patient condition data.

### FIELD

The following relates generally to medical records. More particularly, embodiments herein relate to supplying intervention protocols in response to a multi-layered acute kidney injury (AKI) assessment.

### BACKGROUND

Acute kidney injury (AKI) is a commonly occurring condition in critically ill patients. Developing AKI in an intensive care unit (ICU) typically leads to worse outcomes. For example, developing AKI may result in an increased risk of mortality, longer length of stay, and/or poor quality of life post discharge. Therefore, preventing AKI from occurring or reducing the severity or duration of AKI can positively impact patient outcomes.

AKI management often includes therapy decisions taken to prevent kidney injury and increased monitoring for patients at risk for AKI (or having AKI). Risk of acute kidney injury (AKI) can be due to multiple types of factors. These factors can individually be assessed via various predictive models. Such predictive models can help with early identification of patients at risk of developing AKI, which would enable clinicians to plan and manage these patients better by following safe medication dosing guidelines.

There are several inherent problems with current AKI predictive models. Accordingly, it is currently challenging to use AKI predictive models as a reliable source for real-time AKI management.

The following discloses certain improvements to overcome these problems and others.

### SUMMARY

As discussed above, it is currently challenging to use AKI predictive models as a reliable source for real-time AKI management.

In the present disclosure, procedures are described that combine the outputs from multiple types of AKI assessments to trigger clinical action protocols. Such procedures may be used for other clinical decision support algorithms to link predicted risk scores to potential intervention protocols.

In some implementations herein, AKI risk assessment may be broken down into two portions. In the first portion, AKI risk assessment may be based on a patient's preexisting medical history data (e.g., information regarding comorbidities, medication history, surgical history, medical procedures, patient allergies, diagnoses, reason for current admission, and/or the like) and demographics (e.g., information regarding patient height, weight, gender, ethnicity, blood type, and/or the like). In the second portion, AKI risk assessment may be based on a patient's dynamic clinical state as characterized by labs, vital signs, interventions, etc. These two risk types have different levels of impact and work on different time scales. In addition, these two risk types provide different information to the clinician in management of AKI. The risk from the first category of factors is non-modifiable, therefore clinicians can aim to reduce the general risk by following renal protective medication strategy and increased monitoring. The second category of factors can be modified and therefore these can be used as targets for personalized therapy. As will be described in greater detail below, techniques are described herein for synergistically considering both these two risk types to predict each type of risk in a unified assessment.

Additionally, AKI may be categorized into three stages of varying severity. However, a first group of patients with a risk of a patient developing any AKI stage may be treated in a categorically different way than a second group of patients with a risk of a patient developing moderate-to-severe AKI (e.g., stage 2 or stage 3 AKI). Therefore, different techniques may be utilized to predict the risk for these two different groups of patients.

In some implementations herein, these three AKI risk aspects (e.g., 1) the AKI risk based on preexisting medical history data and demographics, 2) the AKI risk of a patient developing any AKI stage based on dynamic information, and 3) the AKI risk of a patient developing moderate-to-severe AKI based on dynamic information) are integrated to produce a single prediction which clinicians will use to make decisions. Therefore, some techniques described herein may combine the outputs of these three AKI risk aspects in a clinically meaningful way in a real-time clinical deployment for decision making. As will be described in greater detail below, a framework is described to combine three (or more) AKI risk aspect assessments to produce a unified risk score. Each of the three (or more) AKI risk aspect assessments encapsulates a different aspect of AKI risk.

In one aspect, an apparatus is configured to determine whether a baseline AKI risk prediction is above a baseline threshold based on patient demographic data and patient medical history data, and perform a continuous AKI risk prediction. The continuous AKI risk prediction includes determining whether an any risk of AKI prediction is above an any AKI threshold based on dynamic intervention data and/or dynamic patient condition data, and determining an AKI stage prediction in response to a determination that the any risk of AKI prediction is above the any AKI threshold based on the dynamic intervention data and/or the dynamic patient condition data.

In another aspect, a method includes determining whether a baseline AKI risk prediction is above a baseline threshold based on patient demographic data and patient medical history data, and perform a continuous AKI risk prediction. The continuous AKI risk prediction includes determining whether an any risk of AKI prediction is above an any AKI threshold based on dynamic intervention data and/or dynamic patient condition data, and determining an AKI stage prediction in response to a determination that the any risk of AKI prediction is above the any AKI threshold based on the dynamic intervention data and/or the dynamic patient condition data.

In yet another aspect, a machine-readable storage includes machine-readable instructions, which when executed, include operations to determine whether a baseline AKI risk prediction is above a baseline threshold based on patient demographic data and patient medical history data, and perform a continuous AKI risk prediction. The continuous AKI risk prediction includes determining whether an any risk of AKI prediction is above an any AKI threshold based on dynamic intervention data and/or dynamic patient condition data, and determining an AKI stage prediction in response to a determination that the any risk of AKI prediction is above the any AKI threshold based on the dynamic intervention data and/or the dynamic patient condition data.

In still another aspect, an apparatus includes means for determining whether a baseline AKI risk prediction is above a baseline threshold based on patient demographic data and patient medical history data, and perform a continuous AKI risk prediction. The continuous AKI risk prediction includes determining whether an any risk of AKI prediction is above an any AKI threshold based on dynamic intervention data and/or dynamic patient condition data, and determining an AKI stage prediction in response to a determination that the any risk of AKI prediction is above the any AKI threshold based on the dynamic intervention data and/or the dynamic patient condition data. The apparatus also includes means for transferring a notification that intervention is needed.

It should be appreciated that all combinations of the foregoing concepts and additional concepts discussed in greater detail below (provided such concepts are not mutually inconsistent) are contemplated as being part of the subject matter disclosed herein. In particular, all combinations of claimed subject matter appearing at the end of this disclosure are contemplated as being part of the subject matter disclosed herein. It should also be appreciated that terminology explicitly employed herein that also may appear in any disclosure incorporated by reference should be accorded a meaning most consistent with the particular concepts disclosed herein.

These and other aspects of the various embodiments will be apparent from and elucidated with reference to the embodiment(s) described hereinafter.

### BRIEF DESCRIPTION OF THE DRAWINGS

The various advantages of the embodiments will become apparent to one skilled in the art by reading the following specification and appended claims, and by referencing the following drawings, in which:

FIG. 1 is an illustration of a block diagram of an example AKI management system according to an embodiment;

FIG. 2 is an illustration of a flowchart of a method for monitoring and/or management of acute kidney injury (AKI) according to an embodiment;

FIG. 3 is an illustration of a flowchart of a further method for monitoring and/or management of acute kidney injury (AKI) according to an embodiment;

FIG. 4 is an illustration of a flowchart of another method for monitoring and/or management of acute kidney injury (AKI) according to an embodiment;

FIG. 5 is an illustration of a flowchart of a still further method for monitoring and/or management of acute kidney injury (AKI) according to an embodiment;

FIG. 6 is an illustration of a block diagram of a computer program product according to an embodiment;

FIG. 7 is a further illustration of a EMR management system according to an embodiment; and

FIG. 8 is an illustration of a hardware apparatus including a semiconductor package according to an embodiment.

### DETAILED DESCRIPTION

As will be described in greater detail below, in some implementations discussed herein, an automated tool may advantageously be used to address parts of the challenges in using AKI predictive models as a reliable source for real-time AKI decisions.

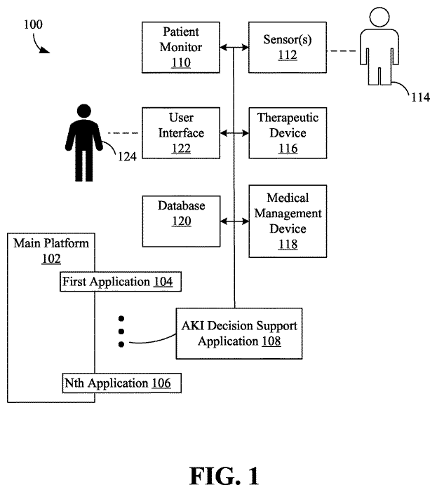

FIG. 1 is an illustration of a block diagram of an example AKI management system 100 according to an embodiment. For example, the AKI management system 100 may be centralized or may be distributed and may include some or all elements and components of one or more computers or computer systems.

In some implementations, the AKI management system 100 may be utilized as a control point element of an Integrated Clinical Environment (ICE). As used herein, the “Integrated Clinical Environment (ICE)” refers to a platform to create a medical Internet of Things (IoT) associated with the care of a patient. In such an implementation, the AKI management system 100 may support many real-time clinical decision support algorithm and closed loop control algorithms of medical devices in the ICE.

In the illustrated implementation, the AKI management system 100 may include a main platform 102. In some implementations, the main platform 102 may be embodied as a server computer or a plurality of server computers (e.g., interconnected to form a server cluster, cloud computing resource, the like, and/or combinations thereof).

In some implementations, a first application 104 though an Nth application 106, which may include an AKI decision support application 108, may be associated with the main platform 102. The operations of the AKI decision support application 108 to trigger AKI notifications and/or clinical interventions will be described in greater detail below.

Additionally, or alternatively, the AKI management system 100 may include a patient monitor 110, a sensor 112 (e.g., one or more sensors 112 that may be associated with a patient 114), a therapeutic device 116, a medical management device 118, a database 120, a user interface 122 (e.g., one or more user interfaces 122 may be associated with a user 124), the like, and/or combinations thereof. For example, the main platform 102, the patient monitor 110, the sensor 112, the therapeutic device 116, the medical management device 118, the database 120, and/or the user interface 122 may be in communication with one another via Internet based communicating, cloud based communication, wired communication, wireless communication, the like, and/or combinations thereof.

In an example, the patient monitor 110 may be utilized to enter patient data to the database 120. For example, the patient monitor 110 may determine measured patient data (e.g., via one of more of the sensors 112). In such an example, the patient monitor 110 may be configured to monitor a patient for vital signs and the like, and the patient monitor 110 may communicate such measured patient data to the database 120. For example, the sensors 112 may determine dynamic patient condition data including patient vitals (e.g., blood pressure, pulse, temperature, respiration, and/or the like) and/or patient test results (e.g., creatine level, urine flow, potassium level, oxygen saturation, blood glucose level, carbon dioxide level, and/or the like).

In some implementations, the patient monitor 110 may comprise a bedside-type monitor, a transport-type monitor, a central station-type monitor, the like, and/or combinations thereof.

In some implementations, the sensors 112 may be minimally invasive-style sensors (e.g., by puncturing the skin, sensing through the skin, the like, and/or combinations thereof). Sensors 112 may be wired or wireless. In implementations where the sensors 112 are wireless, the sensors 112 may have power consumption limitations. Accordingly, operations to increase the patient monitoring frequency (e.g., by increasing data collection frequency and/or data transmission frequency) typically will adversely impact power consumption. Accordingly, it is advantageous to increase the patient monitoring frequency in a limited manner responsive to the needs of each individual patient.

In another example, the therapeutic device 116 may be utilized to enter patient data to the database 120. For example, the therapeutic device 116 may determine measured patient data. In such an example, the therapeutic device 116 may be configured to monitor the delivery of a particular therapy (e.g., a non-medication treatment) to a patient and may communicate such measured patient data to database 120.

In some implementations, the therapeutic device 116 may supply and/or monitor the administration of one or more patient procedures (e.g., dialysis).

In a further example, the medical management device 118 may be utilized to enter patient data to the database 120. For example, the medical management device 118 may determine measured patient data. In such an example, the medical management device 118 may be configured to monitor medication delivery to a patient and may communicate such measured patient data to the database 120.

In some implementations, the medical management device 118 may supply and/or monitor the administration of one or more patient medications (e.g., blood pressure medications, diuretic medications, anti-anemia medications, cholesterol lowering medications, vitamin supplements, and/or the like).

Additionally, or alternatively, in a still further example, the user interface 122 may be utilized to enter patient data to the database 120. In some implementations, user interface 122 may be implemented via one or more form factor devices (e.g., a smart phone, a tablet, a laptop, a workstation, and/or the like), an interface associated with the main platform, and/or an interface associated with the patient monitor 110. Additionally, or alternatively, a care provider (e.g., user 124) may enter measured patient data through an analog device, a non-networked patient monitor, a non-networked therapeutic device, a non-networked medical management device, the like, and/or combinations thereof. In such an example, the care provider may enter such measured patient data to the database 120 via one or more user interfaces 122.

In the illustrated implementation, the database 120 may include one or more types of patient data. For example, the database 120 may include patient data including laboratory result data, microbiology data, medication data, vital sign data, care order data, admission discharge and transfer data, and/or the like. As used herein, the term “database” refers to a collection of data and information organized in such a way as to allow the data and information to be stored, retrieved, updated, and/or manipulated. The term “database” as used herein may also refer to databases that may reside locally or that may be accessed from a remote location (e.g., via remote network servers).

As used herein, the term “patient data” refers to data or information for identifying an individual. Patient data may include measured patient data from an analog medical device, a sensor, a patient monitor, a therapeutic device, a medical management device, a medical imaging device, the like, and/or combinations thereof. Additionally, patient data may include a patient's name, age, weight, previous medical history, admission number, medical personnel in charge, date of admission, medical condition, a medical status, like, and/or combinations thereof.

Additionally, or alternatively, in some implementations, the database 120 may include or be associated with a simulated database. In such an example, such a simulated database may generate estimated patient data. For example, the simulated database may utilize some measured patient data from the patient monitor 110, the sensor 112, the therapeutic device 116, the medical management device 118, and/or the user interface 122 to generate some other estimated patient data. Such a simulated database may utilize digital twin technology to perform the estimation, for example. In such an example, such estimated patient data may be marked to indicate its estimated nature (rather than measured patient data). Additionally, or alternatively, a weight factor may be applied to the estimated patient data so that the estimated patient data may have a lower weight than corresponding measured patient data.

As will be described in greater detail below, several different techniques of triggering AKI notifications and/or clinical interventions may be implemented in the AKI management system 100 via the AKI decision support application 108.

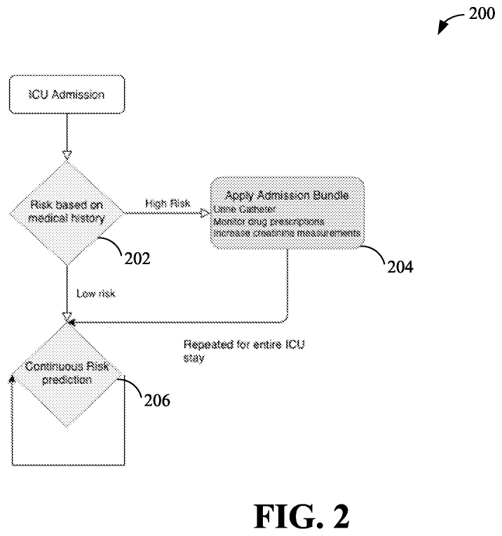

FIG. 2 shows an example method 200 for monitoring and/or management of acute kidney injury (AKI) according to an embodiment. The method 200 may generally be implemented in an AKI management system, such as, for example, the AKI management system 100 (FIG. 1), already discussed.

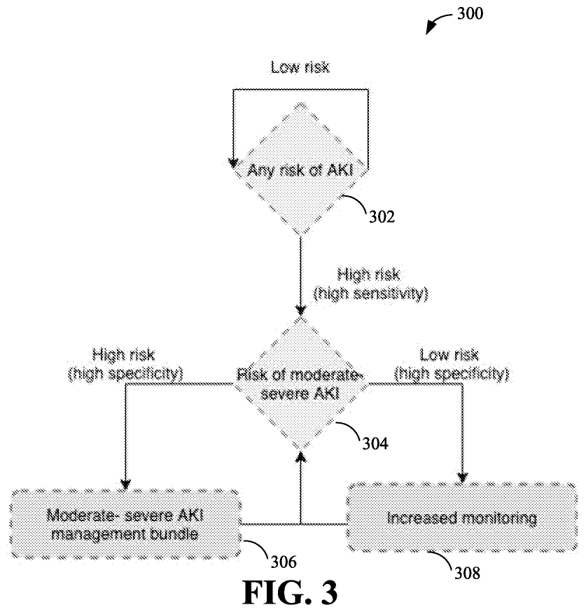

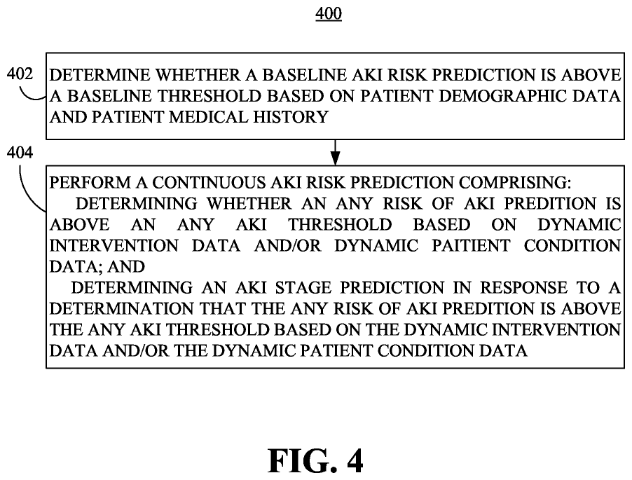

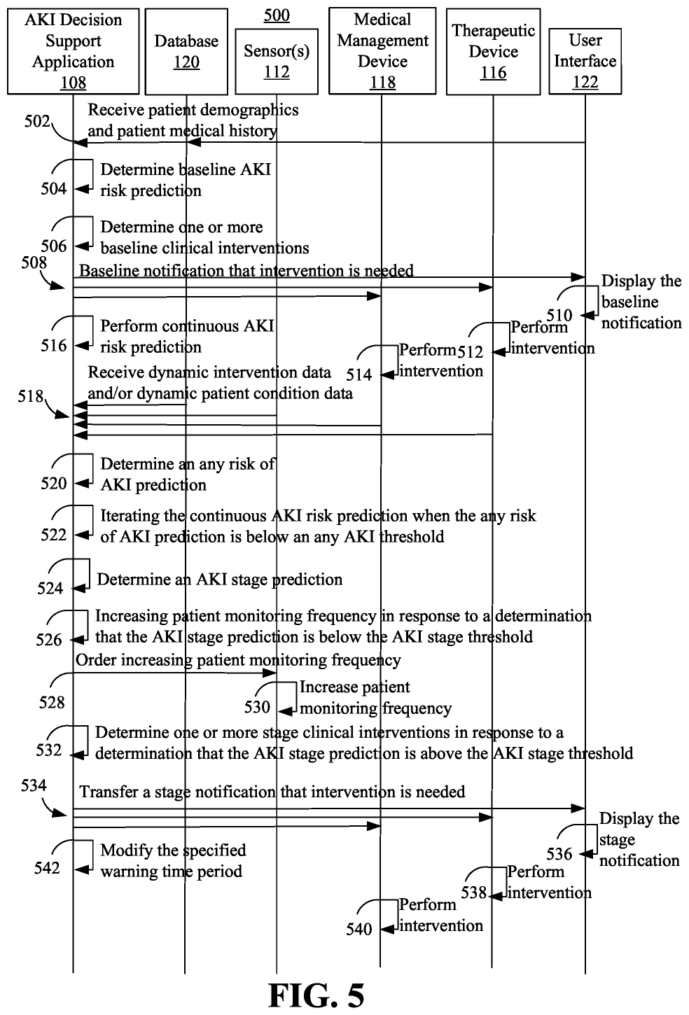

In an embodiment, the method 200 (as well as method 300 (FIG. 3), method 400 (FIG. 4), and/or method 500 (FIG. 5)) may be implemented in logic instructions (e.g., software), configurable logic, fixed-functionality hardware logic, etc., or any combination thereof.

Illustrated processing block 202 provides for determining a risk based on medical history. For example, a baseline AKI risk prediction may be determined based on patient demographic data and patient medical history data. The baseline AKI risk prediction may be compared to a baseline threshold to see if the baseline AKI risk prediction is above an acceptable risk.

Illustrated processing block 204 provides for applying an admission bundle. For example, one or more baseline clinical interventions may be determined in response to a determination that the baseline AKI risk prediction is above the baseline threshold. As illustrated, the one or more baseline clinical interventions may include application of a urine catheter, monitoring drug prescriptions, increased frequency of creatine measurements, and/or the like.

Illustrated processing block 206 provides for performing a continuous AKI risk prediction. For example, a continuous AKI risk prediction may include determining an any risk of AKI prediction based on dynamic intervention data and/or dynamic patient condition data. Illustrated processing block 206 may be repeated for a patient's entire ICU stay.

In operation, method 200 illustrates an overview of multiple AKI risk techniques working together and being linked to clinical protocols. The first AKI risk technique (e.g., baseline AKI risk prediction) is typically applied at ICU admission and uses a patient's demographics and medical history to predict their baseline risk of AKI. Such a baseline AKI risk prediction is based on predetermined static data and therefore remains constant throughout a patient's ICU stay. Based on the Such a baseline AKI risk prediction model and a chosen baseline threshold, patients can be categorized as high baseline risk or low baseline risk. The choice of threshold depends on the tolerance for false positives and risks vs. benefits for missing true cases vs. having too many false positives. These choices are linked to the ‘cost’ of the intervention. This ‘cost’ may include the financial cost of the intervention/protocol as well as the ‘cost’ of missed treatment (e.g., the potential for harm if treatment was not started on time). These costs can be quantified using previously published literature to determine the baseline threshold (e.g., an optimal threshold). High risk patients (e.g., as determined by the baseline threshold) can be put on an enhanced monitoring bundle, renal protective medication strategy, etc.

As will be described in greater detail below, the performance of the continuous AKI risk prediction (e.g., processing block 206) is used to quantify the risk of AKI from dynamic interventions and observations during an ICU stay. These include risk factors such as creatinine, blood pressure, medications, surgery, etc. This risk is continuously changing as new dynamic observations become available. These processes can start running once sufficient data from labs, vitals, medications, etc. have been obtained. Additionally, or alternatively, these processes recalculate the risk score at a pre-determined frequency (e.g., hourly, when new data is available, etc.). Continuous risk score prediction can be split into two different threshold determinations as shown below in FIG. 3.

Additional and/or alternative operations for method 200 are described in greater detail below in the description of FIG. 5.

FIG. 3 shows an example method 300 for monitoring and/or management of acute kidney injury (AKI) according to an embodiment. The method 300 may generally be implemented in an AKI management system, such as, for example, the AKI management system 100 (FIG. 1), already discussed.

As discussed above, method 300 of FIG. 3 provides additional details regarding the performance of the continuous AKI risk prediction (e.g., processing block 206 from method 200). As will be described in greater detail below, method 300 splits the continuous AKI risk prediction into two associated prediction tasks with two decision thresholds and clinical protocols linked together.

Illustrated processing block 302 provides for determining an any risk of AKI prediction. For example, the any risk of AKI prediction may be determined based on the dynamic intervention data and/or the dynamic patient condition data. In some implementations, the any risk of AKI prediction may be compared to an any AKI threshold to determine whether the any risk of AKI prediction is above the any risk of AKI threshold.

In some implementations, the any AKI threshold is associated with a risk of developing any AKI within a specified warning time period.

As illustrated, the performance of the continuous AKI risk prediction may be iterated. For example, the performing of the continuous AKI risk prediction is iterated in response to a determination that the any risk of AKI prediction is below the any AKI threshold.

In operation, processing block 302 calculates the risk of developing any AKI stage. For example, a model may be trained as a classification problem with patients separated into those who develop AKI and those who don't develop AKI. Since the model makes predictions in advance of the actual event, the model is trained using data measured up to ‘p’ hours before AKI development. The parameter ‘p’ is the number of hours of advanced warning the model is trained to give. An example value of ‘p’ is 6 hours (e.g., the model predicts the risk of developing AKI 6 hours prior to event). Any classification framework such as logistic regression, boosting, the like, and/or combinations thereof can be used to train the algorithm. A final model architecture choice may be made based on the prediction task and/or the available input data. Data samples may be classified as high or low predicted risk of AKI based on a threshold. (e.g., an any risk of AKI threshold). Since the goal of this procedure is to identify as many as possible cases which might develop AKI and the drawback of having false positives is minimal, the any risk of AKI threshold may be picked to maximize sensitivity at the risk of sacrificing specificity. If the risk score exceeds a preset threshold (e.g., the any risk of AKI threshold) then the next technique (e.g., processing block 302, which predicts risk of moderate-severe AKI) may be run.

In some embodiments, the risk of developing AKI at any stage may be calculated using a model trained using historical data. In a related embodiment, a model to predict the onset of AKI can be trained using retrospective prospective evaluation, which can use historical data and known results of that historical data, such as the data preceding the onset of AKI injury in past patients. By doing so, the model can be tested on data collected prior to the onset of AKI injuries in one or more patient to determine whether the model rises above a threshold of accuracy in predicting the onset of AKI when compared to what actually occurred. For example, a model to predict the onset of AKI can be applied to data preceding the onset of AKI associated with 1000 patients from a period of July 2000 to July 2010 to determine whether the model falls within an acceptable threshold of accuracy in determining AKI onset.

Illustrated processing block 304 provides for determining risk of moderate-severe AKI (e.g., an AKI stage prediction). For example, the AKI stage prediction is determined in response to a determination that the any risk of AKI prediction is above the any AKI threshold. In some implementations, the AKI stage prediction may be compared to an AKI stage threshold to determine whether the AKI stage prediction is above an AKI stage threshold.

Illustrated processing block 306 provides for determining a moderate-severe AKI management bundle (e.g., determining one or more stage clinical interventions). For example, one or more stage clinical interventions may be determined in response to a determination that the AKI stage prediction is above the AKI stage threshold.

In some implementations, the stage threshold may include a first stage threshold associated with a first stage clinical intervention and a second stage threshold associated with a second stage clinical intervention. For example, the first stage threshold provides a different intervention than the second stage threshold. Accordingly, individual clinical intervention are capable of being considered to take into account variations in the ‘costs’ (e.g., monetary cost, adverse health risks, and/or the like) and benefits (e.g., improved health potential) on an intervention-by intervention basis.

Illustrated processing block 308 provides for increasing patient monitoring frequency. For example, increasing the patient monitoring frequency may be done in response to a determination that the AKI stage prediction is below the AKI stage threshold.

In operation, processing block 304 calculates the risk of developing moderate-severe AKI. For this model, the training problem may be setup differently (e.g., only patients who develop moderate-severe AKI (e.g., Stage 2 and above)) are classified as a positive group and patients who don't develop moderate-severe AKI are classified as a negative/control group. The parameter ‘p’ can also be changed in this algorithm which would change the number of hours of advanced warning. An example of this would be a 12 hour advanced warning (e.g., a larger ‘p’ value may be chosen to reflect the fact that moderate-severe AKI takes longer to develop and the therapeutic targets also take longer to change). Therefore, a longer advanced warning period allows clinicians to intervene efficiently. The final model architecture can be selected from available classification algorithms, custom developed algorithms, the like, and/or combinations thereof. The final model architecture can be the same or similar model architecture as the any AKI model. The output of the final model architecture may be the predicted risk of developing moderate-severe AKI and data samples may be classified based on a threshold (e.g., a stage threshold). The action linked to the prediction of this model may be targeted therapy, increased monitoring, etc. Therefore, the stage threshold may be selected such that the stage threshold has high specificity (e.g., low false positives) to ensure that only patients who will benefit from the intervention receive such interventions. Patients who don't meet the stage threshold will continue to be monitored as previously and their risk score updated.

Additionally, or alternatively, the procedures presented above can be applied to other conditions such as acute respiratory distress syndrome (ARDS), acute decompensated heart failure (ADHF), and more. To abstract this framework, the first step for any condition is detecting a baseline risk using prior demographics and medical history only. This first step would stratify patients who are at higher risk for certain conditions (e.g., such as ARDS, ADHF, etc.) so their cases can be managed differently from the general ICU population. Once additional dynamic measurements are obtained in the ICU, a continuous risk model (or more than one) can continuously update the risk and once this risk prediction crosses a threshold, further preventative and therapeutic actions can be taken. The thresholds can be adjusted for each condition depending on the prevalence of the condition and the ‘cost’ of the action. For example, the ‘cost’ refers to a balance between benefits derived from the actions and the risks from the actions. Some actions are low risk (such as more frequent lab measurements) while some other actions (such as intubation) are associated with high risk. Therefore, the thresholds which trigger various actions can be dependent on the action associated with it on an action-by-action basis. Such procedures provide a framework to link machine learning based algorithm predictions to actions (e.g., interventions).

Additionally, or alternatively, the procedures described herein may provide a framework for clinical deployment of decision support algorithms. These procedures can work together with many clinical decision support (CDS) algorithms (such as AKI, ARDS, ADHF, HSI etc.). Clinical decision support (CDS) refers to computer-based support of clinical staff responsible for making decisions for the care of patients. Computer-based support for clinical decision-making staff may take many forms, from patient-specific visual/numeric health status indicators to patient-specific health status predictions and patient-specific health care recommendations. Further, the procedures described herein may be deployed on analytics platforms (such as NGCAP, ICCA, IBE, etc.) in conjunction with CDS algorithms.

Additional and/or alternative operations for method 300 are described in greater detail below in the description of FIG. 5.

FIG. 4 shows an example method 400 for monitoring and/or management of acute kidney injury (AKI) according to an embodiment. The method 400 may generally be implemented in an AKI management system, such as, for example, the AKI management system 100 (FIG. 1), already discussed.

Illustrated processing block 402 provides for determining a baseline AKI risk prediction. For example, a baseline AKI risk prediction may be determined based on patient demographic data and patient medical history data. The baseline AKI risk prediction may be compared to a baseline threshold to see if the baseline AKI risk prediction is above an acceptable risk. For example, such patient demographic data may include information regarding patient height, weight, gender, ethnicity, blood type, and/or the like. Further, the patient medical history data may include information regarding comorbidities, medication history, surgical history, medical procedures, patient allergies, diagnoses, reason for current admission and/or the like.

Illustrated processing block 404 provides for performing a continuous AKI risk prediction. For example, a continuous AKI risk prediction may include determining an any risk of AKI prediction based on dynamic intervention data and/or dynamic patient condition data. The any risk of AKI prediction may be compared to an any AKI threshold see if there is any risk of AKI. Additionally, the continuous AKI risk prediction may include determining an AKI stage prediction in response to a determination that the any risk of AKI prediction is above the any AKI threshold. The AKI stage prediction may be based on the dynamic intervention data and/or the dynamic patient condition data.

For example, such dynamic intervention data may include patient medications (e.g., blood pressure medications, diuretic medications, anti-anemia medications, cholesterol lowering medications, vitamin supplements, the like, and/or combinations thereof), patient procedures (e.g., dialysis), and/or the like. Further, dynamic patient condition data may include patient vitals (e.g., blood pressure, pulse, temperature, respiration, and/or the like) and/or patient test results (creatine level, urine flow, potassium level, oxygen saturation, blood glucose level, carbon dioxide level, and/or the like).

In some examples, the methods described herein (e.g., method 200, method 300, method 400, and/or method 500) may be performed at least in part by cloud processing.

Additional and/or alternative operations for method 400 are described in greater detail below in the description of FIG. 5.

FIG. 5 is a flowchart of an example of another method 500 for monitoring and/or management of acute kidney injury (AKI) according to an embodiment. The method 500 may generally be implemented in an AKI management system, such as, for example, the EMR management system 100 (FIG. 1), already discussed.

In an embodiment, the method 500 (as well as method 200 (FIG. 2), method 300 (FIG. 3), and/or method 400 (FIG. 4)) may be implemented in logic instructions (e.g., software), configurable logic, fixed-functionality hardware logic, etc., or any combination thereof. While certain portions of an AKI management system are illustrated in method 500, other portions of the AKI management system 100 (FIG. 1) have been intentionally left out to simplify the explanation of the method.

Illustrated processing block 502 provides for receiving patient demographic data and patient medical history data. For example, the patient demographic data and patient medical history data may be received by the AKI decision support application 108 from database 102. Additionally, or alternatively, the patient demographic data and patient medical history data may be received by the AKI decision support application 108 from user interface 122.

As described above, patient demographic data may include information regarding patient height, weight, gender, ethnicity, blood type, and/or the like. Additionally, patient medical history data may include comorbidities, medication history, surgical history, medical procedures, patient allergies, diagnoses, reason for current admission, and/or the like.

Illustrated processing block 504 provides for determining a baseline AKI risk prediction. For example, the baseline AKI risk prediction may be based on the patient demographic data and the patient medical history data. The baseline AKI risk prediction may be compared to a baseline threshold to determine whether the baseline AKI risk prediction is above the baseline threshold.

For example, such patient demographic data may include information regarding patient height, weight, gender, ethnicity, blood type, and/or the like. Further, the patient medical history data may include information regarding patient allergies, diagnoses, medical procedures, and/or the like.

In some implementations, the baseline threshold may include a first baseline threshold associated with a first baseline clinical intervention and a second baseline threshold associated with a second baseline clinical intervention. For example, the first baseline threshold provides a different intervention than the second baseline threshold. Accordingly, individual clinical intervention are capable of being considered to take into account variations in the ‘costs’ (e.g., monetary cost, adverse health risks, and/or the like) and benefits (e.g., improved health potential) on an intervention-by intervention basis.

Illustrated processing block 506 provides for determining one or more baseline clinical interventions. For example, the one or more baseline clinical interventions may be determined in response to a determination that the baseline AKI risk prediction is above the baseline threshold.

Illustrated processing block 508 provides for transferring a baseline notification that the one or more baseline clinical interventions is needed. For example, the baseline notification that the one or more baseline clinical interventions is needed may be transferred to the user interface 122 associated with a care provider.

Additionally, or alternatively, the baseline notification that the one or more baseline clinical interventions is needed may be transferred to the therapeutic device 116 and/or the medical management device 118. In such an example, the AKI decision support application 108 may order an automated administration of at least one of the one or more baseline clinical interventions via one or more of the therapeutic devices 118, and/or order an automated administration of at least one of the one or more baseline clinical interventions via one or more of the medical management devices 116.

Illustrated processing block 510 provides for displaying the baseline notification. For example, the baseline notification may be displayed via the user interface 122 in response to the baseline notification that the one or more baseline clinical interventions is needed. As discussed above, in some implementations, user interface 122 may be implemented via one or more form factor devices (e.g., a smart phone, a tablet, a laptop, a workstation, and/or the like), an interface associated with the main platform, and/or an interface associated with the patient monitor 110.

Illustrated processing block 512 provides for performing one or more interventions. For example, one or more interventions may be performed via the therapeutic device 116 in response to the baseline notification that the one or more baseline clinical interventions is needed.

For example, the therapeutic device 116 may supply and/or monitor the administration of dialysis treatments.

Illustrated processing block 514 provides for performing one or more interventions. For example, one or more interventions may be performed via the medical management device 118 in response to the baseline notification that the one or more baseline clinical interventions is needed.

For example, the medical management device 118 may supply and/or monitor the administration of one or more patient medications (e.g., blood pressure medications, diuretic medications, anti-anemia medications, cholesterol lowering medications, vitamin supplements, and/or the like).

Illustrated processing block 516 provides for performing a continuous AKI risk prediction. For example, a continuous AKI risk prediction may include determining an any risk of AKI prediction based on dynamic intervention data and/or dynamic patient condition data. The any risk of AKI prediction may be compared to an any AKI threshold see if there is any risk of AKI. As will be described in greater detail below, the continuous AKI risk prediction may include determining an AKI stage prediction in response to a determination that the any risk of AKI prediction is above the any AKI threshold. The AKI stage prediction may be based on the dynamic intervention data and/or the dynamic patient condition data.

For example, such dynamic intervention data may include patient medications (e.g., blood pressure medications, diuretic medications, anti-anemia medications, cholesterol lowering medications, vitamin supplements, the like, and/or combinations thereof), patient procedures (e.g., dialysis), and/or the like. Further, dynamic patient condition data may include patient vitals (e.g., blood pressure, pulse, temperature, respiration, and/or the like) and/or patient test results (creatine level, urine flow, potassium level, oxygen saturation, blood glucose level, carbon dioxide level, and/or the like).

Illustrated processing block 518 provides for receiving dynamic intervention data and/or dynamic patient condition data. For example, the dynamic intervention data and/or dynamic patient condition data may be received from one or more of the database 120, the sensor(s) 112, the medical management device 11, the therapeutic device 116, and/or user interface 122.

In some implementations, the dynamic intervention data may include patient medications (e.g., blood pressure medications, diuretic medications, anti-anemia medications, cholesterol lowering medications, vitamin supplements, and/or the like) and patient procedures (e.g., surgeries, dialysis, and/or the like) Similarly, the dynamic patient condition data may include patient vitals (e.g., blood pressure, pulse, temperature, respiration, and/or the like) and/or patient test results (e.g., creatine level, urine flow, potassium level, oxygen saturation, blood glucose level, carbon dioxide level, and/or the like).

Illustrated processing block 520 provides for determining an any risk of AKI prediction. For example, the any risk of AKI prediction may be determined based on the dynamic intervention data and/or the dynamic patient condition data. In some implementations, the any risk of AKI prediction may be compared to an any AKI threshold to determine whether the any risk of AKI prediction is above the any risk of AKI threshold.

In some implementations, the any AKI threshold is associated with a risk of developing any AKI within a specified warning time period.

Illustrated processing block 522 provides for iterating the performing of the continuous AKI risk prediction. For example, the performing of the continuous AKI risk prediction is iterated in response to a determination that the any risk of AKI prediction is below the any AKI threshold.

Illustrated processing block 524 provides for determining an AKI stage prediction. For example, the AKI stage prediction is determined in response to a determination that the any risk of AKI prediction is above the any AKI threshold. In some implementations, the AKI stage prediction may be compared to an AKI stage threshold to determine whether the AKI stage prediction is above an AKI stage threshold.

Illustrated processing block 526 provides for increasing patient monitoring frequency. For example, increasing the patient monitoring frequency may be done in response to a determination that the AKI stage prediction is below the AKI stage threshold.

Illustrated processing block 528 provides for ordering an increased patient monitoring frequency. For example, the order for increased patient monitoring frequency may be transferred to the sensor(s) 112 from AKI decision support application 108.

In some implementations, the operation to increase the patient monitoring frequency includes an order to increase data collection frequency and/or data transmission frequency. Such operations may impact a power supply associated with the sensor(s) 112 (e.g., in situations where wireless sensors are utilized); accordingly, it is advantageous to proportionally throttle data collection frequency and/or data transmission frequency in response to the changing needs of a patient.

Illustrated processing block 530 provides for increasing patient monitoring frequency. For example, the sensor(s) 112 may increase the patient monitoring frequency in response to the order for increased patient monitoring frequency from AKI decision support application 108.

Illustrated processing block 532 provides for determining one or more stage clinical interventions. For example, one or more stage clinical interventions may be determined in response to a determination that the AKI stage prediction is above the AKI stage threshold.

In some implementations, the stage threshold may include a first stage threshold associated with a first stage clinical intervention and a second stage threshold associated with a second stage clinical intervention. For example, the first stage threshold provides a different intervention than the second stage threshold. Accordingly, individual clinical intervention are capable of being considered to take into account variations in the ‘costs’ (e.g., monetary cost, adverse health risks, and/or the like) and benefits (e.g., improved health potential) on an intervention-by intervention basis.

Illustrated processing block 534 provides for transferring a stage notification that the one or more stage clinical interventions is needed. For example, the stage notification that the one or more stage clinical interventions is needed may be transferred to the user interface 122 associated with a care provider.

Additionally, or alternatively, the stage notification that the one or more stage clinical interventions is needed may be transferred to the therapeutic device 116 and/or the medical management device 118. In such an example, the AKI decision support application 108 may order an automated administration of at least one of the one or more stage clinical interventions via one or more of the therapeutic devices 118, and/or order an automated administration of at least one of the one or more stage clinical interventions via one or more of the medical management devices 116.

Illustrated processing block 536 provides for displaying the stage notification. For example, the stage notification may be displayed via the user interface 122 in response to the stage notification that the one or more stage clinical interventions is needed.

Illustrated processing block 538 provides for performing one or more interventions. For example, one or more stage interventions may be performed via the therapeutic device 116 in response to the stage notification that the one or more stage clinical interventions is needed.

For example, the therapeutic device 116 may supply and/or monitor the administration of dialysis treatments.

Illustrated processing block 540 provides for performing one or more interventions. For example, one or more interventions may be performed via the medical management device 118 in response to the stage notification that the one or more stage clinical interventions is needed.

For example, the medical management device 118 may supply and/or monitor the administration of one or more patient medications (e.g., blood pressure medications, diuretic medications, anti-anemia medications, cholesterol lowering medications, vitamin supplements, and/or the like).

Illustrated processing block 542 provides for modifying the specified warning time period. For example, the specified warning time period may be modified in response to one or more of the determination that the AKI stage prediction is below the AKI stage threshold, the determination that the AKI stage prediction is above the AKI stage threshold, and the determination that the any risk of AKI prediction is above the any AKI threshold.

It will be appreciated that some or all of the operations in method 500 above that have been described using a “pull” architecture (e.g., polling for new information followed by a corresponding response) may instead be implemented using a “push” architecture (e.g., sending such information when there is new information to report), and vice versa.

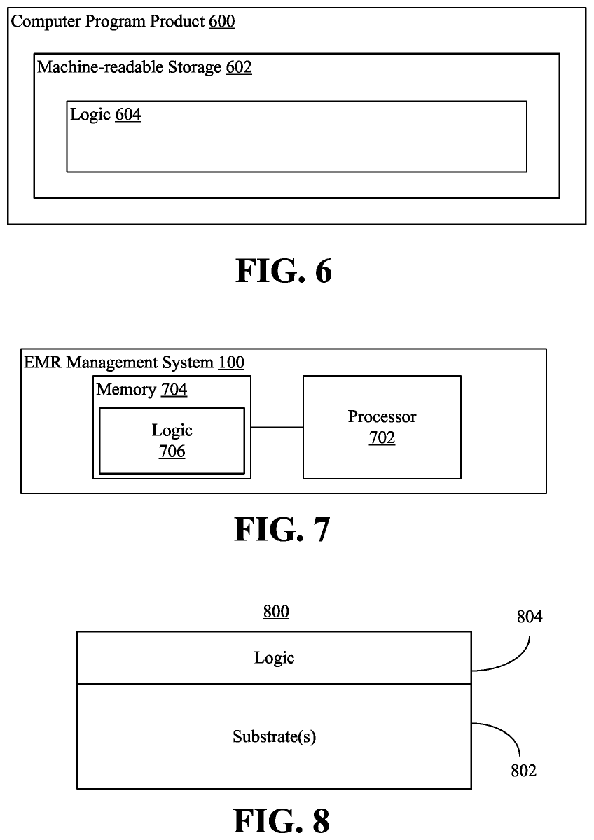

FIG. 6 illustrates a block diagram of an example computer program product 600. In some examples, as shown in FIG. 6, computer program product 600 includes a machine-readable storage 602 that may also include logic 604. In some implementations, the machine-readable storage 602 may be implemented as a non-transitory machine-readable storage. In some implementations the logic 604 may be implemented as machine-readable instructions, such as software, for example. In an embodiment, the logic 604, when executed, implements one or more aspects of the method 200 (FIG. 2), the method 300 (FIG. 3), the method 400 (FIG. 4), the method 500 (FIG. 1), and/or realize the AKI management system 100 (FIG. 1), already discussed.

FIG. 7 shows an illustrative example of the AKI management system 100. In the illustrated example, the AKI management system 100 may include a processor 702 and a memory 704 communicatively coupled to the processor 702. The memory 704 may include logic 706 as a set of instructions. In some implementations the logic 706 may be implemented as software. In an embodiment, the logic 706, when executed by the processor 702, implements one or more aspects of the method 200 (FIG. 2), the method 300 (FIG. 3), the method 400 (FIG. 4), the method 500 (FIG. 1), and/or realize the AKI management system 100 (FIG. 1), already discussed.

In some implementations, the processor 702 may include a general purpose controller, a special purpose controller, a storage controller, a storage manager, a memory controller, a micro-controller, a general purpose processor, a special purpose processor, a central processor unit (CPU), the like, and/or combinations thereof.

Further, implementations may include distributed processing, component/object distributed processing, parallel processing, the like, and/or combinations thereof. For example, virtual computer system processing may implement one or more of the methods or functionalities as described herein, and the processor 702 described herein may be used to support such virtual processing.

In some examples, the memory 704 is an example of a computer-readable storage medium. For example, memory 704 may be any memory which is accessible to the processor 702, including, but not limited to RAM memory, registers, and register files, the like, and/or combinations thereof. References to “computer memory” or “memory” should be interpreted as possibly being multiple memories. The memory may for instance be multiple memories within the same computer system. The memory may also be multiple memories distributed amongst multiple computer systems or computing devices.

FIG. 8 shows an illustrative semiconductor apparatus 800 (e.g., chip and/or package). The illustrated apparatus 800 includes one or more substrates 802 (e.g., silicon, sapphire, or gallium arsenide) and logic 804 (e.g., configurable logic and/or fixed-functionality hardware logic) coupled to the substrate(s) 802. In an embodiment, the logic 804 implements one or more aspects of the method 200 (FIG. 2), the method 300 (FIG. 3), the method 400 (FIG. 4), the method 500 (FIG. 1), and/or realize the AKI management system 100 (FIG. 1), already discussed.

In some implementations, logic 804 may include transistor array and/or other integrated circuit/IC components. For example, configurable logic and/or fixed-functionality hardware logic implementations of the logic 804 may include configurable logic such as, for example, programmable logic arrays (PLAs), field programmable gate arrays (FPGAs), complex programmable logic devices (CPLDs), or fixed-functionality logic hardware using circuit technology such as, for example, application specific integrated circuit (ASIC), complementary metal oxide semiconductor (CMOS) or transistor-transistor logic (TTL) technology, the like, and/or combinations thereof.

All definitions, as defined and used herein, should be understood to control over dictionary definitions, definitions in documents incorporated by reference, and/or ordinary meanings of the defined terms.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. The term “coupled” may be used herein to refer to any type of relationship, direct or indirect, between the components in question, and may apply to electrical, mechanical, fluid, optical, electromagnetic, electromechanical, or other connections. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components.

In the claims, as well as in the specification above, the terms “first”, “second”, etc. may be used herein only to facilitate discussion, and carry no particular temporal or chronological significance unless otherwise indicated.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

The indefinite articles “a” and “an,” as used herein in the specification and in the claims, unless clearly indicated to the contrary, should be understood to mean “at least one.”

As used herein, the term “or” or “and/or” is inclusive and not exclusive, unless expressly indicated otherwise or indicated otherwise by context. Therefore, herein, “A or B” means “A, B, or both,” unless expressly indicated otherwise or indicated otherwise by context. Moreover, “and” is both joint and several, unless expressly indicated otherwise or indicated otherwise by context. Therefore, herein, “A and B” means “A and B, jointly or severally,” unless expressly indicated otherwise or indicated otherwise by context.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

As used in this application and in the claims, a list of items joined by the term “one or more of” may mean any combination of the listed terms. For example, the phrases “one or more of A, B or C” may mean A; B; C; A and B; A and C; B and C; or A, B and C.

As is described above in greater detail, one or more processor, other unit, the like, and/or combinations thereof may fulfill the functions of several items recited in the claims.

As is described above in greater detail, a computer program may be stored/distributed on a suitable medium, such as an optical storage medium or a solid-state medium supplied together with or as part of other hardware, but may also be distributed in other forms, such as via the Internet or other wired or wireless telecommunication systems.

It should also be understood that, unless clearly indicated to the contrary, in any methods discussed herein that include more than one step or act, the order of the steps or acts of the method is not necessarily limited to the order in which the steps or acts of the method are recited. Further, such methods may include additional or alternative steps or acts. As used in the claims, the mere fact that certain measures are recited in mutually different dependent claims does not indicate that a combination of these measured cannot be used to advantage.

It is also noted that the claims may include reference signs/numerals in accordance with PCT Rule 6.2(b). However, the present claims should not be considered to be limited to the exemplary embodiments corresponding to the reference signs/numerals.

Those skilled in the art will appreciate from the foregoing description that the broad techniques of the embodiments of the present invention can be implemented in a variety of forms. Therefore, while the embodiments of this invention have been described in connection with particular examples thereof, the true scope of the embodiments of the invention should not be so limited since other modifications will become apparent to the skilled practitioner upon a study of the drawings, specification, and following claims.

1. An apparatus (100), comprising:
a user interface (122); and
a patient monitor (110) communicatively coupled to the user interface (122), the patient monitor (110) to:
determine whether a baseline AKI risk prediction is above a baseline threshold based on patient demographic data and patient medical history data;
perform a continuous AKI risk prediction, the continuous AKI risk prediction comprising:
determine whether an any risk of AKI prediction is above an any AKI threshold based on dynamic intervention data and/or dynamic patient condition data; and
determine an AKI stage prediction in response to a determination that the any risk of AKI prediction is above the any AKI threshold based on the dynamic intervention data and/or the dynamic patient condition data.

2. The apparatus (100) of claim 1, wherein the baseline threshold includes a first baseline threshold associated with a first baseline clinical intervention and a second baseline threshold associated with a second baseline clinical intervention, and wherein the first baseline threshold is different than the second baseline threshold.

3. The apparatus (100) of claim 1, wherein the patient monitor (110) is further to:
receive the patient demographic data and the patient medical history data; and
determine one or more baseline clinical interventions in response to a determination that the baseline AKI risk prediction is above the baseline threshold.

4. The apparatus (100) of claim 3, wherein the patient monitor (110) is further to: transfer a baseline notification that the one or more baseline clinical interventions is needed to the user interface (122), order an automated administration of at least one of the one or more baseline clinical interventions via one or more therapeutic devices (118), and/or order an automated administration of at least one of the one or more baseline clinical interventions via one or more medical management devices (116).

5. The apparatus (100) of claim 1, wherein the continuous AKI risk prediction further comprises operations to:
receive the dynamic intervention data and/or the dynamic patient condition data, wherein the dynamic intervention data comprises patient medications and patient procedures, and wherein the dynamic patient condition data comprises patient vitals and/or patient test results; and
iterate the performing the continuous AKI risk prediction in response to a determination that the any risk of AKI prediction is below the any AKI threshold,
wherein the any AKI threshold is associated with a risk of developing any AKI within a specified warning time period.

6. The apparatus (100) of claim 5, wherein the AKI stage prediction further comprises operations to:
determine whether the AKI stage prediction is above an AKI stage threshold based on the dynamic intervention data and/or the dynamic patient condition data; and
determine one or more stage clinical interventions in response to a determination that the AKI stage prediction is above the AKI stage threshold.

7. The apparatus (100) of claim 6, wherein the AKI stage prediction further comprises operations to:
increase patient monitoring frequency in response to a determination that the AKI stage prediction is below the AKI stage threshold, wherein the operation to increase the patient monitoring frequency comprises an order to one or more sensors (112) to increase data collection frequency and/or data transmission frequency.

8. The apparatus (100) of claim 6, wherein the AKI stage prediction further comprises operations to:
transfer a stage notification that the one or more stage clinical interventions is needed to the user interface (122), order an automated administration of at least one of the one or more stage clinical interventions via one or more therapeutic devices (118), and/or order an automated administration of at least one of the one or more stage clinical interventions via one or more medical management devices (116).

9. The apparatus (100) of claim 6, wherein the AKI stage threshold includes a first AKI stage threshold associated with a first AKI stage clinical intervention and a second AKI stage threshold associated with a second AKI stage clinical intervention, and wherein the first AKI stage threshold is different than the second AKI stage threshold.

10. The apparatus (100) of claim 6, wherein the continuous AKI risk prediction further comprises operations to:
modify the specified warning time period in response to one or more of the determination of whether the AKI stage prediction is above an AKI stage threshold and the determination of whether the any risk of AKI prediction is above the any AKI threshold.

11. A method (500), comprising:
determining whether a baseline AKI risk prediction is above a baseline threshold based on patient demographic data and patient medical history data;
performing a continuous AKI risk prediction, the continuous AKI risk prediction comprising:
determining whether an any risk of AKI prediction is above an any AKI threshold based on dynamic intervention data and/or dynamic patient condition data; and
determining an AKI stage prediction in response to a determination that the any risk of AKI prediction is above the any AKI threshold based on the dynamic intervention data and/or the dynamic patient condition data.

12. The method (500) of claim 11, further comprising:
receiving the patient demographic data and the patient medical history data;
determining one or more baseline clinical interventions in response to a determination that the baseline AKI risk prediction is above the baseline threshold, wherein the baseline threshold includes a first baseline threshold associated with a first baseline clinical intervention and a second baseline threshold associated with a second baseline clinical intervention, and wherein the first baseline threshold is different than the second baseline threshold; and
transferring a baseline notification that the one or more baseline clinical interventions is needed to the user interface (122), ordering an automated administration of at least one of the one or more baseline clinical interventions via one or more therapeutic devices (118), and/or ordering an automated administration of at least one of the one or more baseline clinical interventions via one or more medical management devices (116).

13. The method (500) of claim 11, wherein the continuous AKI risk prediction further comprises:
receiving the dynamic intervention data and/or the dynamic patient condition data, wherein the dynamic intervention data comprises patient medications and patient procedures, and wherein the dynamic patient condition data comprises patient vitals and/or patient test results; and
iterating the performing the continuous AKI risk prediction in response to a determination that the any risk of AKI prediction is below the any AKI threshold,
wherein the any AKI threshold is associated with a risk of developing any AKI within a specified warning time period.

14. The method (500) of claim 13, wherein the AKI stage prediction further comprises:
determining whether the AKI stage prediction is above an AKI stage threshold based on the dynamic intervention data and/or the dynamic patient condition data; and
determining one or more stage clinical interventions in response to a determination that the AKI stage prediction is above the AKI stage threshold.

15. The method (500) of claim 14, wherein the AKI stage prediction further comprises:
increasing patient monitoring frequency in response to a determination that the AKI stage prediction is below the AKI stage threshold, wherein increasing the patient monitoring frequency comprises an order to one or more sensors (112) to increase data collection frequency and/or data transmission frequency;
transferring a stage notification that the one or more stage clinical interventions is needed to the user interface (122), ordering an automated administration of at least one of the one or more stage clinical interventions via one or more therapeutic devices (118), and/or ordering an automated administration of at least one of the one or more stage clinical interventions via one or more medical management devices (116); and
modifying the specified warning time period in response to one or more of the determination of whether the AKI stage prediction is above an AKI stage threshold and the determination of whether the any risk of AKI prediction is above the any AKI threshold,
wherein the AKI stage threshold includes a first AKI stage threshold associated with a first AKI stage clinical intervention and a second AKI stage threshold associated with a second AKI stage clinical intervention, and wherein the first AKI stage threshold is different than the second AKI stage threshold.

16. At least one computer readable medium (602), comprising a set of instructions (604), which when executed by a computing device, cause the computing device to:
determine whether a baseline AKI risk prediction is above a baseline threshold based on patient demographic data and patient medical history data;
perform a continuous AKI risk prediction, the continuous AKI risk prediction comprising operations to:
determine whether an any risk of AKI prediction is above an any AKI threshold based on dynamic intervention data and/or dynamic patient condition data; and
determine an AKI stage prediction in response to a determination that the any risk of AKI prediction is above the any AKI threshold based on the dynamic intervention data and/or the dynamic patient condition data.

17. The at least one computer readable medium (602) of claim 16, wherein the set of instructions, which when executed by the computing device, cause the computing device further to:
receive the patient demographic data and the patient medical history data;
determine one or more baseline clinical interventions in response to a determination that the baseline AKI risk prediction is above the baseline threshold, wherein the baseline threshold includes a first baseline threshold associated with a first baseline clinical intervention and a second baseline threshold associated with a second baseline clinical intervention, and wherein the first baseline threshold is different than the second baseline threshold; and
transfer a baseline notification that the one or more baseline clinical interventions is needed to the user interface (122), order an automated administration of at least one of the one or more baseline clinical interventions via one or more therapeutic devices (118), and/or order an automated administration of at least one of the one or more baseline clinical interventions via one or more medical management devices (116).

18. The at least one computer readable medium (602) of claim 16, wherein the continuous AKI risk prediction further comprises operations to:
receive the dynamic intervention data and/or the dynamic patient condition data, wherein the dynamic intervention data comprises patient medications and patient procedures, and wherein the dynamic patient condition data comprises patient vitals and/or patient test results; and
iterate the performing the continuous AKI risk prediction in response to a determination that the any risk of AKI prediction is below the any AKI threshold,
wherein the any AKI threshold is associated with a risk of developing any AKI within a specified warning time period.

19. The at least one computer readable medium (602) of claim 18, wherein the AKI stage prediction further comprises operations to:
determine whether the AKI stage prediction is above an AKI stage threshold based on the dynamic intervention data and/or the dynamic patient condition data; and
determine one or more stage clinical interventions in response to a determination that the AKI stage prediction is above the AKI stage threshold.

20. The at least one computer readable medium (602) of claim 19, wherein the AKI stage prediction further comprises operations to:
increase patient monitoring frequency in response to a determination that the AKI stage prediction is below the AKI stage threshold, wherein the operation to increase the patient monitoring frequency comprises an order to one or more sensors (112) to increase data collection frequency and/or data transmission frequency;
transfer a stage notification that the one or more stage clinical interventions is needed to the user interface (122), order an automated administration of at least one of the one or more stage clinical interventions via one or more therapeutic devices (118), and/or order an automated administration of at least one of the one or more stage clinical interventions via one or more medical management devices (116); and
modify the specified warning time period in response to one or more of the determination of whether the AKI stage prediction is above an AKI stage threshold and the determination of whether the any risk of AKI prediction is above the any AKI threshold,
wherein the AKI stage threshold includes a first AKI stage threshold associated with a first AKI stage clinical intervention and a second AKI stage threshold associated with a second AKI stage clinical intervention, and wherein the first AKI stage threshold is different than the second AKI stage threshold.
